(19) United States
(12) Patent Application Publication
(10) Pub. No.: US 2021/0345964 A1
(43) Pub. Date: Nov. 11, 2021

(54) BIOLOGICAL INFORMATION MEASURING DEVICE

(71) Applicant: SOSAIKOUSEIKAI CLINICAL FOUNDATION MATSUNAMI RESEARCH PARK, Gifu (JP)

(72) Inventor: Hidetoshi Matsunami, Gifu (JP)

(21) Appl. No.: 17/379,203

(22) Filed: Jul. 19, 2021

Related U.S. Application Data
(63) Continuation of application No. PCT/JP2020/001235, filed on Jan. 16, 2020.

(30) Foreign Application Priority Data
Jan. 18, 2019 (JP) 2019-006650

(51) Int. Cl. A61B 5/00; G01N 33/49

(52) U.S. Cl. CPC A61B 5/6833; A61B 5/14552; A61B 5/4839; G01N 33/4925

(57) ABSTRACT

Provided is a biological information measuring device with wearability for satisfying the measurement precision needed in a home medical support system. Also provided is a biological information measuring device with wearability for enhancing the QOL of a user. The present invention provides a biological information measuring device provided with a sensor and an adhesive member having an opening which surrounds the sensor. A measuring surface of the sensor is exposed. The sensor may be disposed so as to be separated from the adhesive member. The sensor includes a reflection-type pulse oximeter, and the thickness of the adhesive member from the surface of the biological information measuring device body on which the adhesive member is disposed may be greater than the thickness from the surface of the biological information measuring device body to an end part of the reflection-type pulse oximeter.

### CROSS-REFERENCE TO RELATED APPLICATIONS

This application is a continuation of International Application No. PCT/JP2020/001235, filed on Jan. 16, 2020, which claims priority to Japanese Patent Application No. 2019-006650, filed on Jan. 18, 2019, the entire contents of which are incorporated herein by reference.

### FIELD

The present invention relates to a biological information measuring device. An embodiment of the present invention relates particularly to a biological information measuring device attached to a user's skin and continuously measuring biological information.

### BACKGROUND

The necessity and importance of home medical care have been increasing more and more in recent years from the viewpoint of independent living and Quality of Life (QOL). Construction of the support system is indispensable for home medical care, and various support systems and devices used for them have been proposed. In Japanese Patent No. 5795424 and Japanese Patent No. 5917754, the present inventors provided a home medical support system which realizes effective medical support using a wearable terminal in which a collection device which is mounted to a living body of a person to be supported and collects biological information from the living body of the person to be supported and a wireless transmission unit which wirelessly transmits the biological information collected by the collection device are incorporated. To detect the abnormality of the person to be supported and provide prompt support, the biological information of the person to be supported which is collected by the wearable terminal must be highly accurate.

In light of the increasing health-consciousness in recent years, there has been proposed a system in which biological information acquired by a wearable terminal is used to support a user's health management and effective exercise (International patent publication No. WO2016/036582). In addition, for the purpose of continuously collecting biological information, a wearable terminal in which power consumption is reduced has been proposed (Japanese laid-open patent publication No. 2017-176827). However, the wearable terminals described in International patent publication No. WO2016/036582 and Japanese laid-open patent publication No. 2017-176827 have wearability during exercise and fashion properties because they are intended to support exercise, but do not have wearability which satisfies the measurement accuracy required for a system for medical use such as a home medical support system.

In addition, Japanese laid-open patent publication No. 2017-080141 describes a respiratory analysis device for monitoring a patient with sleep apnea syndrome, a biological sensor device capable of being attached to and detached from a human body via an adhesive member, and an electronic device such as a wearable terminal are combined. However, Japanese laid-open patent publication No. 2017-080141 does not examine the wearability which satisfies the measurement accuracy required for the system for medical use.

### SUMMARY

One of the objects of the present invention is to provide a biological information measuring device having wearability capable of obtaining high measurement accuracy. Further, one of the objects of the present invention is to provide a biological information measuring device having wearability for improving QOL of a user.

According to an embodiment of the present invention, there is provided a biological information measuring device including sensors and an adhesive member having an opening surrounding the sensors, wherein measuring surfaces of the sensors are exposed.

The sensors may be disposed so as to be separated from the adhesive member.

The sensors include a reflection-type pulse oximeter, and a thickness of the adhesive member from a surface of the biological information measuring device body on which the adhesive member is disposed may be greater than a thickness from the surface of the biological information measuring device body to an end part of the reflection-type pulse oximeter.

The adhesive member is disposed on a surface of the biological information measuring device body opposite to a surface on which the sensor is disposed, and the adhesive member may have an adhesiveness on a surface on the same side as the surface on which the sensor is disposed.

The adhesive member may have vents.

The adhesive member may contain one or more additive agents selected from a group consisting of an antibacterial agent, an aromatic agent, capsaicin, and methyl salicylate.

The biological information measuring device may further include an input unit, and a communication unit, the input unit may be disposed on the surface opposite to the surface on which the sensor is disposed.

The biological information measuring device may be attached to an abdomen of a user, and the biological information measuring device may be supplied with power from a belt worn by the user.

The biological information measuring device may be attached to the back of the user, and the biological information measuring device may be supplied with power from a seat seated by the user.

### BRIEF DESCRIPTION OF THE DRAWINGS

FIG. 1A is a schematic view showing a biological information measuring device 100 according to an embodiment of the present invention and is a perspective view showing the top surface of the biological information measuring device 100;

FIG. 1B is a perspective view showing the bottom surface of a biological information measuring device 100;

FIG. 2 is a block diagram showing a biological information measuring device body 110 according to an embodiment of the present invention;

FIG. 3A is a schematic diagram for explaining an attachment position of the biological information measuring device 100, and is a schematic diagram showing the attachment position of the biological information measuring device 100 as viewed from the front of a user 1;

FIG. 3B is a schematic diagram showing the attachment position of the biological information measuring device 100 as viewed from the side of a user 1,

FIG. 4A is a schematic diagram showing a charging method of the biological information measuring device 100 and shows a wired charging method of a biological information measuring device 100c attached to an abdomen 2;

FIG. 4B shows a wireless charging method of the biological information measuring device 100c attached to the abdomen 2;

FIG. 5 shows a charging method of a biological information measuring device 100d attached to the back;

FIG. 6A is a schematic diagram for explaining a biological information measuring device 200 in which a plurality of sensors is disposed;

FIG. 6B is cross-sectional view in a line segment AA′ in FIG. 6A;

FIG. 7A is a schematic view showing a biological information measuring device 300 according to an embodiment of the present invention and is a perspective view showing a top surface of the biological information measuring device 300;

FIG. 7B is a cross-sectional view in a line segment AA′ in FIG. 7A;

FIG. 8A is a schematic view showing a biological information measuring device 400 according to an embodiment of the present invention and is a perspective view showing a top surface of the biological information measuring device 400;

FIG. 8B is a cross-sectional view in a line segment AA′ in FIG. 8A;

FIG. 9 is a schematic diagram showing a home medical support system 10 according to an embodiment of the present invention;

FIG. 10 is a schematic diagram showing a safe driving assistance system 10A according to an embodiment of the present invention;

FIG. 11 is a block diagram showing a safe driving assistance in-vehicle device 510 according to an embodiment of the present invention; and

FIG. 12 is a schematic diagram showing a health management system 10B according to an embodiment of the present invention.

### DESCRIPTION OF EMBODIMENTS

Hereinafter, a biological information measuring device and a home medical support system according to the present invention will be described with reference to the drawings. The biological information measuring device and the home medical support system according to the present invention are not to be construed as being limited to the description contents of the embodiments described below. In the drawings referred to in this embodiment, the same portions or portions having similar functions are denoted by the same reference numerals, and a repetitive description thereof is omitted.

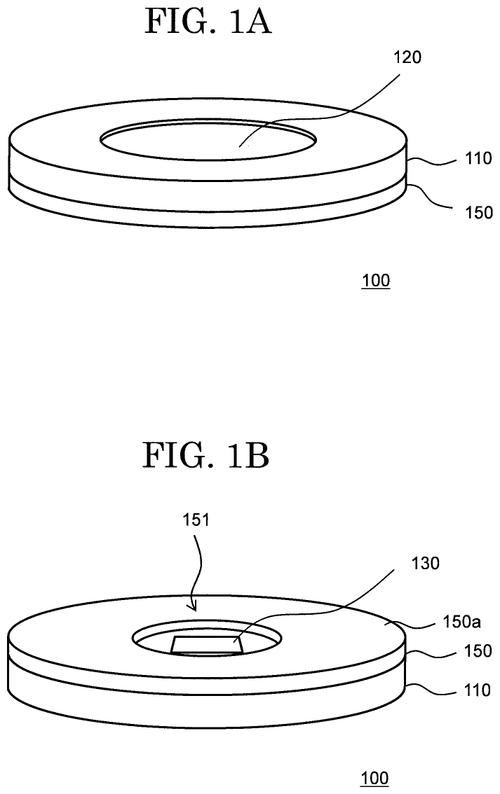

FIG. 1 is a schematic diagram showing a biological information measuring device 100 according to an embodiment of the present invention. FIG. 1A is a perspective view showing a top surface of the biological information measuring device 100, and FIG. 1B is a perspective view showing a bottom surface of the biological information measuring device 100. The biological information measuring device 100 is a sheet-shaped or flat plate-shaped device. The biological information measuring device 100 is attached to a body surface of a user, and continuously acquires biological information of the user. Here, “continuously acquires biological information” is not intended only to continuously acquire biological information of the user but is also intended to intermittently acquire biological information of the user at a predetermined time or a predetermined interval of time. In FIG. 1, a disk-shaped biological information measuring device 100 is shown as an example, but as long as the biological information measuring device 100 has the sheet-shaped or flat plate-shaped shape, the shape the of the biological information measuring device 100 viewed from the top may be a rectangle, polygon, rectangle or polygon with L-shaped corners, elliptical shape, or other shapes.

The biological information measuring device 100 includes, for example, a biological information measuring device body 110 in which a sensor 130 for measuring biological information of a user is disposed, and an adhesive member 150 disposed on the same surface as a surface of the biological information measuring device body 110 on which the sensor 130 is disposed. As will be also described in the embodiment described later, the adhesive member 150 may be disposed on a surface of the biological information measuring device body 110 opposite to the surface on which the sensor 130 is disposed. In this embodiment, the adhesive member 150 has an opening 151 surrounding the sensor 130. Therefore, the adhesive member 150 is not disposed on a measuring surface of the sensor 130, and the measuring surface of the sensor 130 is exposed from the adhesive member 150.

The adhesive member 150 is a member for attaching the biological information measuring device body 110 to a body surface (skin) of the user and has an adhesiveness. Although a known adhesive agent for medical use used in surgical tape, a bandage, or the like can be used for the adhesive member 150, it is preferable to use a skin-friendly adhesive agent. The adhesive member 150 can be selected from, for example, but not limited to, a urethane-based adhesive, a hydrophobic acrylic adhesive, a hydrophilic acrylic adhesive, a silicone-based adhesive, a rubber-based adhesive, a synthetic rubber-based adhesive, and the like. The rubber-based adhesive may be a raw rubber. Further, the adhesive member 150 may be a gel sheet containing these materials.

In addition, a skin-friendly skin protective agent can be used for the adhesive member 150. For example, a karaya-based skin protective agent, a carboxy methylcellulose (CMC)-based skin protective agent or a mixed-type skin protective agent can be used for the adhesive member 150. The karaya-based skin protective agent is a skin protective agent mainly composed of natural karaya gum. The karaya skin protective agent has a weak adhesive strength, but it becomes softer and more easily deformed when the temperature increases. Therefore, the Karaya-based skin protective agent is soft and has a followability which adheres to skin, is rich in absorbency, and has antibacterial property. The CMC-based skin protective agent is a skin protective agent in which CMC, pectin, and gelatin are blended as a hydrophilic polymer, and polyisobutylene is blended as a hydrophobic polymer. In the CMC-based skin protective agent, the hydrophilic polymer is responsible for absorption of sweat and liquid excrement, and the hydrophobic polymer is responsible for enhancing the adhesiveness. The CMC-based skin protective agent has a long usable period due to its strong adhesive strength and stability against temperature and low absorbency. The mixed-type skin protective agent is a skin protective agent in which gelatin and polyisobutylene are blended in karaya gum. The mixed-type skin protective agent contains karaya and has a good affinity for skin compared with the CMC-based skin protectant, and the mixed-type skin protective agent can provide good skin management.

As the adhesive agent 150, examples of the karaya-based skin protective agent, the carboxymethylcellulose (CMC)-based skin protective agent, and the mixed skin protective agent include the following protective agents, but the adhesive agent is not limited thereto. A KG-based skin protective agent (karaya gum), a CKG-based skin protective agent (a mixture of karaya gum and CMC), a KPB-based skin protective agent (a mixture of karaya gum and pectin as hydrophilic polymers and polyisobutylene as a hydrophobic polymer), a KPBS-based skin protective agent (a mixture of karaya gum and pectin as hydrophilic polymers and polyisobutylene and styrene-isobutylene-styrene as hydrophobic polymers), a CPB-based skin protective agent (a mixture of CMC and pectin as a hydrophilic polymer and polyisobutylene as a hydrophobic polymer), a CPS-based skin protectant (a mixture of CMC and pectin as hydrophilic polymers and styrene-isobutylene-styrene as a hydrophobic polymer), a CPBS-based skin protective agent (a mixture of CMC and pectin as hydrophilic polymers and polyisobutylene and ethylene-acetic acid-vinyl copolymer as hydrophobic polymers), a CPBM-based skin protective agent (a mixture of CMC and pectin as hydrophilic polymers and polyisobutylene and microfiber as hydrophobic polymers), a CPBHS-based skin protective agent (a mixture of CMC and pectin as hydrophilic polymers and polyisobutylene and hydrogenated styrene as hydrophobic polymers), a CPFB-based skin protective agent (a mixture of CMC, pectin and cotton fibers as hydrophilic polymers and polyisobutylene as a hydrophobic polymer), a CB-based skin protective agent (a mixture of CMC as a hydrophilic polymer and polyisobutylene as a hydrophobic polymer), a CS-based skin protective agent (a mixture of CMC as a hydrophilic polymer and styrene-isobutylene-styrene as a hydrophobic polymer), and a CBS-based skin protective agent (a mixture of CMC as a hydrophilic polymer and polyisobutylene and styrene-isobutylene-styrene as a hydrophobic polymer).

In FIG. 1, the adhesive member 150 has the same shape (the shape of the outer peripheral portion as viewed from the bottom surface) and the same size (the size of the outer peripheral portion as viewed from the bottom surface including the opening 151) as the biological information measuring device body 110, but the present embodiment is not limited thereto. If the biological information measuring device body 110 can be attached to the body surface of the user, the adhesive member 150 may be set smaller than the biological information measuring device body 110 in consideration of the elasticity of the adhesive member 150. As described above, since the biological information measuring device 100 includes the adhesive member 150, even if the user who has the biological information measuring device 100 attached thereto moves, the sensor 130 and the body surface of the user maintain a certain distance, so that the measurement sensitivity can be improved.

In an embodiment, the adhesive member 150 may contain one or more additive agents selected from a group consisting of antibacterial agent, an aromatic agent, capsaicin, and methyl salicylate. As the antibacterial agent, a known antibacterial agent contained in an external preparation for medical use can be used. By containing an antimicrobial agent in the adhesive member 150, when the biological information measuring device 100 is attached to the body surface, the growth of bacteria in contact surface with the adhesive member 150 can be suppressed and the inflammation of the skin can be reduced. In addition, a known aromatic agent contained in a medicine and an external preparation can be used for the aromatic agent. A volatile aromatic agent released into the air from the adhesive member 150 due to body temperature is preferred. By containing an aromatic agent in the adhesive member 150, when the biological information measuring device 100 is attached to the body surface, the aromatic agent is released into the air from the adhesive member 150 due to body temperature. Thus, it is also possible to reduce the stress of the user by applying the biological information measuring device 100. In addition, according to the preference of the user, the aromatic agent may be added to the adhesive member 150 to provide a variation in the aromatic agent. That is, the user may be allowed to select the biological information measuring device 100 having a favorite scent. Phytoncide with antibacterial properties and aromatic properties can also be used.

Further, a warm-sensitive effect can be obtained by containing capsaicin in the adhesive member 150. On the other hand, a cool-sensitive effect can be obtained by containing methyl salicylate in the adhesive member 150. Since these are also used in a wet compress, these components may be combined with the above-mentioned antibacterial agent and/or aromatic agent and added to the adhesive member 150.

In an embodiment, the adhesive member 150 is preferably a member which does not transmit light. For example, a colorant may be further added to the above material of the adhesive member 150. In this specification, “does not transmit light” means that the light transmittance is 10% or less, preferably 1% or less, and more preferably 0.1% or less. Since the adhesive member 150 does not transmit light, when the sensor 130 is a sensor measured by an optical means such as a pulsimeter or a reflection-type pulse oximeter, the effect of light from the outside can be suppressed, and the measuring accuracy can be improved.

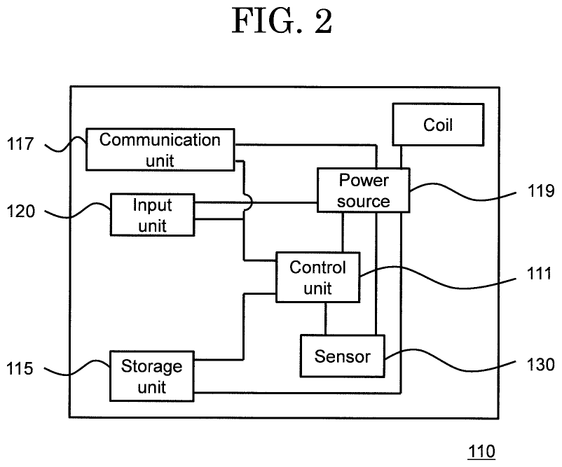

FIG. 2 is a block diagram showing the biological information measuring device body 110 according to an embodiment of the present invention. The biological information measuring device body 110 includes, for example, but not limited to, a control unit 111, a storage unit 115, a communication unit 117, a power source 119, an input unit 120, and the sensor 130.

In an embodiment, the control unit 111 is a device which controls the biological information measuring device body 110, for example, a central processing unit (CPU). The control unit 111 includes a program for controlling the biological information measuring device body 110. The program for controlling the biological information measuring device body 110 is stored in the storage unit 115 and executed by the control unit 111. In an embodiment, the control unit 111 may include an operating system (OS) which controls the biological information measuring device body 110 and an application program or module which functions in the biological information measuring device 100.

In an embodiment, the storage unit 115 may store user information. The user information may include, for example, information which identifies the user, such as the user's authentication code (ID). The storage unit 115 may store a program for controlling the biological information measuring device body 110. The storage unit 115 may temporarily store biological information measured by the sensor 130.

In an embodiment, the communication unit 117 is a device which communicates with the biological information measuring device 100 and an external device. The communication unit 117 includes, for example, a communication means conforming to a wireless communication standard such as Wi-Fi® (a communication means using IEEE 802.11 standard) and Bluetooth®, but is not limited thereto. A physical address (MAC address) is assigned to the communication unit 117, and the biological information measuring device 100 can be identified from the physical address. Since the physical address of the biological information measuring device 100 is associated with the user, the user can be identified based on the physical address of the biological information measuring device 100. As will be described later, in an embodiment, the biological information measuring device 100 can transmit the biological information of the user measured by the sensor 130 to a server including in the home medical support system via the wireless communication by the communication unit 117.

In an embodiment, the power source 119 is a common battery and is a power source which supplies power to each device disposed in the biological information measuring device 100. As will be described in the embodiment described later, the power source 119 may include a connecting terminal and may be charged by supplying power from the outside. The power source 119 may also be charged by supplying power from the outside via wireless power transmission in a non-contact manner. The power source 119 is preferably light in weight and has a large capacity but is not particularly limited.

In an embodiment, as shown in FIG. 1A, the input unit 120 may be disposed on a surface opposite to the surface on which the sensor 130 is disposed in the biological information measuring device 100. The input unit 120 may be, for example, an emergency notification button. For example, the user may be notified that urgent support is required by pressing the input unit 120. As shown in the embodiment described later, the input unit 120 may not be disposed in the biological information measuring device body 110.

In an embodiment, the sensor 130 is a device for acquiring biological information from a user, for example, one or more of measurement devices such as a thermometer, a pulsimeter, a sphygmomanometer, a reflection-type pulse oximeter, and an electrocardiograph may be selected and arranged. In the present embodiment, the pulse rate measured by the pulsimeter is treated as substantially the same as the heart rate. The number, shape, and arrangement of the sensor 130 included in the biological information measuring device 100 may be arbitrarily selected and are not particularly limited. Since a known sensor can be used for a measuring device such as a thermometer, a pulsimeter, a sphygmomanometer, a reflection-type pulse oximeter, and an electrocardiograph used as the sensor 130, a detailed description thereof is omitted.

Also, in an embodiment, as shown in FIG. 1B, the sensor 130 may be arranged separated from the adhesive member 150. In an embodiment, a thickness of the adhesive member 150 from a surface of the biological information measuring device body 110 on which the adhesive member 150 is disposed may be larger than a thickness from the surface of the biological information measuring device body 110 to an end part of the sensor 130. In an embodiment, if the sensor 130 is, for example, a pulsimeter or a reflection-type pulse oximeter, a distance to the surface of the body from the pulsimeter or the reflection-type pulse oximeter required for measurement can be adjusted by the thickness of the adhesive member 150. Preferably, the opening 151 is sized to separate the adhesive member 150 from the sensor 130 so that light used for measurement by the pulsimeter, or the reflection-type pulse oximeter is not diffusely reflected by the adhesive member 150.

### Attachment Position of Biological Information Measuring Device 100

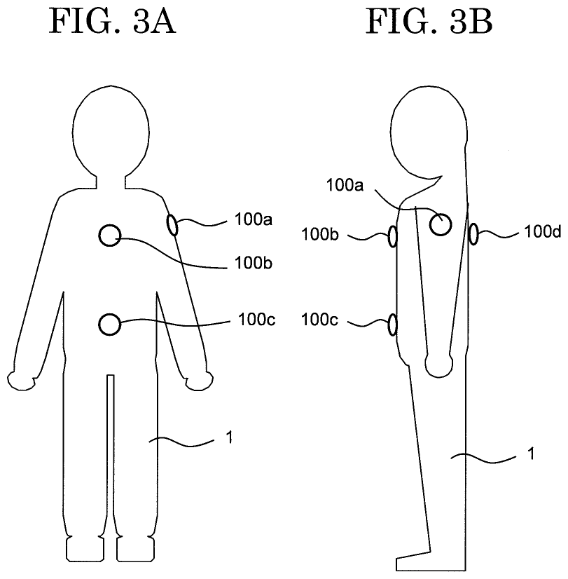

FIG. 3 is a schematic diagram for explaining an attachment position of the biological information measuring device 100. FIG. 3A is a schematic diagram showing the attachment position of the biological information measuring device 100 as viewed from a front surface of a user 1, and FIG. 3B is a schematic diagram showing the attachment position of the biological information measuring device 100 as viewed from a side surface of the user 1. The attachment position of the biological information measuring device 100 is not particularly limited as long as it is a part capable of acquiring biological information of the user 1 but is preferably attached to a part easily attached by the user 1. For example, a biological information measuring device 100a is shown as an example of attaching to an upper arm of the user 1. When the biological information measuring device 100 has the input unit 120, it is preferable that the biological information measuring device 100 is attached to the upper arm opposite to the dominant hand of the user 1. By attaching the biological information measuring device 100 to such a part, when the user 1 feels abnormal in his/her own physical condition, by pressing the input unit 120, the user can send an emergency notification to, for example, a server disposed on the system using the biological information measuring device 100. A biological information measuring device 100b is shown as an example of attaching to the chest of the user 1. By attaching the biological information measuring device 100b to the chest of the user 1, the measurement accuracy by the electrocardiograph is improved, and the reliability of the electrocardiogram is improved.

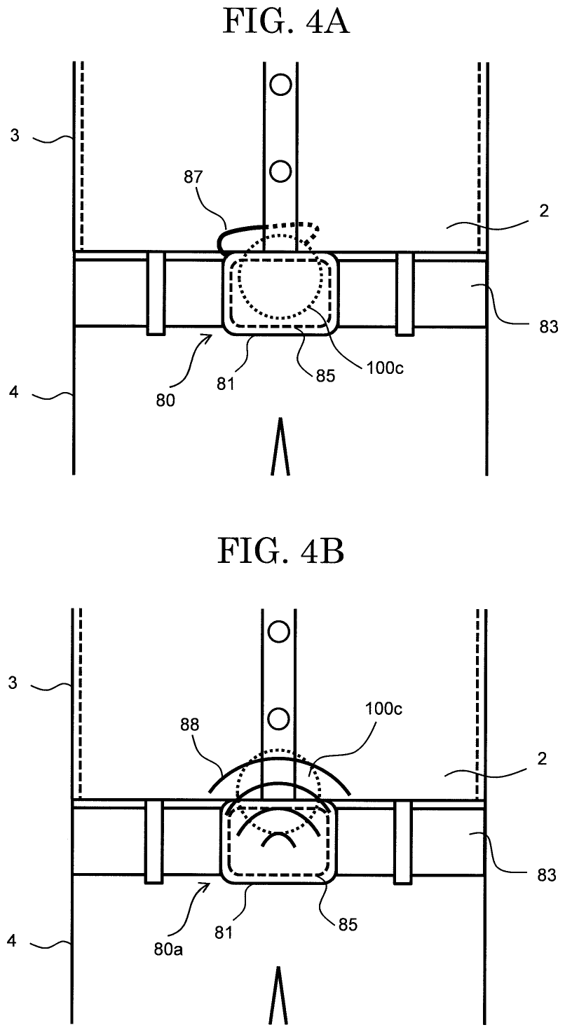

FIG. 4 is a schematic diagram for explaining a charging method of the biological information measuring device 100. A biological information measuring device 100c shown in FIG. 3A and FIG. 3B is shown as an example of attaching to an abdomen 2 of the user 1, and FIG. 4A shows a wiring charging method of the biological information measuring device 100c attached to the abdomen 2. In FIG. 4A, the user 1 wears a shirt 3 and pants 4 and attaches a belt 80 to the pants 4. The belt 80 includes a buckle 81 with a power source 85 and a belt body 83. The buckle 81 is fixed to the abdomen 2 of the user 1 by the belt body 83. The biological information measuring device 100c attached to the abdomen of the user 1 is located on the back side of the buckle 81. Power can be supplied from the power source 85 to the biological information measuring device 100c by connecting the power source 85 and the biological information measuring device 100c by a connector 87. The connector 87 is, for example, an electric wiring, and may be connected to the biological information measuring device 100c through a space between the left front body and the right front body of the shirt 3. The connector 87 may be connected to the biological information measuring device 100c from under the hem of the shirt 3 when the shirt 3 is a cloth to be untucked over, such as a T-shirt.

In FIG. 4B, a belt 80a includes the buckle 81 with the power source 85 and the belt body 83. A power transmission coil connected to the power source 85 is disposed in the buckle 81 of the belt 80a. In an embodiment, a reception coil connected to the power source 119 is disposed in the biological information measuring device 100c. The biological information measuring device 100c is charged from the power source 85 of the belt 80a by means of wireless inductive charging 88. As described above, by attaching the biological information measuring device to the abdomen 2 of the user 1, power can be supplied from the belt worn by the user 1 to the biological information measuring device 100c via the wired or wireless connection.

The power source 85 disposed on the buckle 81 may be a replaceable battery cell or a battery which can be repeatedly charged and discharged. The power source 85 may be connected to an outlet to charge at the time when the user does not wear the belt, such as during sleeping. A solar cell may be disposed on a surface of the buckle 81 to charge the power source 85 by sunlight or indoor lighting. The present embodiment is not limited thereto, and for example, the power source 85 may be disposed on a hat, shoes, jacket, or the like worn by the user 1, and power may be supplied to the biological information measuring device 100c by wire or wirelessly.

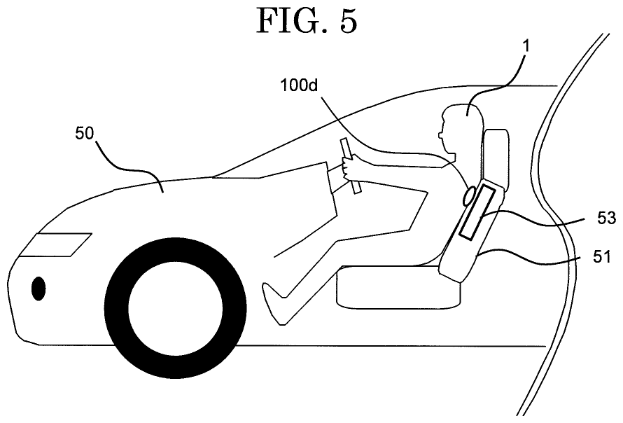

The biological information measuring device 100d shown in FIG. 3B shows an example of attaching to a back of the user 1, and FIG. 5 shows a charging method of the biological information measuring device 100d attached to the back. The user 1 has the biological information measuring device 100d attached to the back. For example, if the user 1 is driving a vehicle 50, a power transmission coil 53 is disposed on a backrest of a seat 51 seated by the user 1. The biological information measuring device 100d attached to the back of the user 1 is arranged to face the power transmission coil 53. By arranging the power transmitting coil 53 on the backrest of the seat 51 and arranging the reception coil connected to the power source 119 on the biological information measuring device 100d, it is possible to perform the wireless inductive charging. The power transmission coil 53 may be supplied with power from a battery to be mounted on the vehicle 50. As described above, by attaching the biological information measuring device 100d to the back of the user 1, power can be supplied from the seat 51 seated by the user 1 to the biological information measuring device 100d. In the present embodiment, an example of charging from the seat 51 disposed in the vehicle 50 has been described, but the charging method is not limited thereto. For example, the biological information measuring device 100d may be charged from a home-use power source by arranging the power transmission coils 53 in chairs, beds, or the like in the home of the user 1.

### Arrangement of Sensors

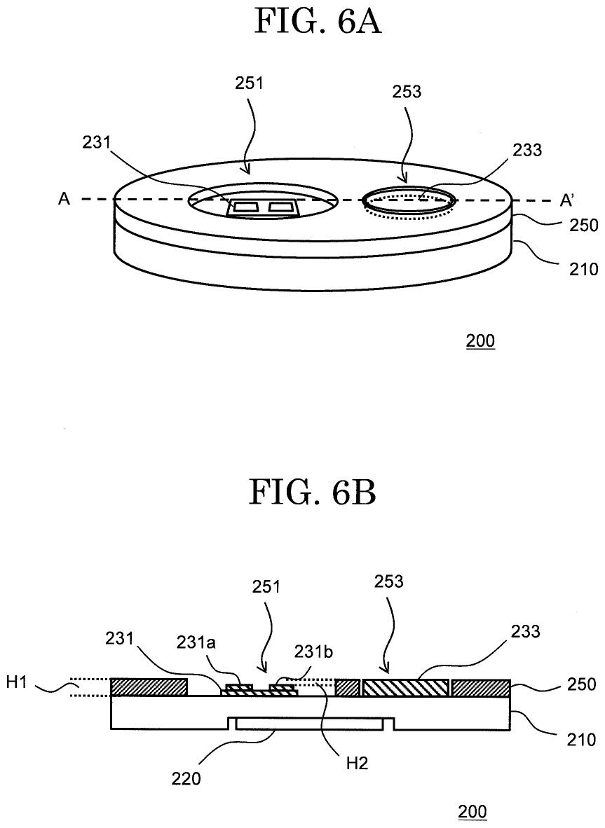

FIG. 6A is a schematic diagram for explaining a biological information measuring device 200 in which a plurality of sensors is arranged, and FIG. 6B shows a cross-sectional view in a line segment AA′ in FIG. 6A. The biological information measuring device 200 includes a sensor 231 selected from, for example, a pulse wave meter or a reflection-type pulse oximeter, and a sensor 233. The biological information measuring device 200 includes an adhesive member 250 disposed on the same surface as a surface of the biological information measuring device body 210 on which the sensors 231 and 233 are disposed. One or more of measurement devices such as a thermometer, a sphygmomanometer, and an electrocardiograph can be selected and arranged as the sensor 233. The sensor 231 has a light-emitting unit 231a and a light-receiving unit 231b. The light-emitting unit 231a is an LED, and the light-receiving unit 231b is a phototransistor. Light is irradiated from the light-emitting unit 231a to skin of the user 1, and the light-receiving unit 231b receives the light reflected by a blood vessel. For this reason, the light-emitting unit 231a and the light-receiving unit 231b are arranged so as to be separated from each other by a distance required to transmit and reflect the light irradiated from the light-emitting unit 231a through the blood vessel and enter the light-receiving unit 231b. When the sensor 231 is a pulsimeter, the light-emitting unit 231a emits, for example, green light with a wavelength near 550 nm. When the sensor 231 is a reflection-type pulse oximeter, the light-emitting unit 231a emits, for example, red light with a wavelength in the vicinity of 660 nm or near-infrared light with a wavelength in the vicinity of 940 nm.

As described above, when the sensor 231 is a pulsimeter or a reflection-type pulse oximeter, the light-receiving unit 231b is separated from the light-emitting unit 231a by a distance required for receiving the light which is irradiated from the light-emitting unit 231a to the skin of the user 1 and reflected through the blood vessel. Therefore, a thickness H1 of the adhesive member 250 is set so that the light-emitting unit 231a separates from the body surface by a distance H2 required for the light-receiving unit 231b to receive the light irradiated from the light-emitting unit 231a to the skin of the user 1 and reflected through the blood vessel. In consideration of the elasticity of the adhesive member 250, the thickness H1 of the adhesive member 250 may be set slightly thicker so that the distance from the light-receiving unit 231b to the skin becomes the distance H2 when the biological information measuring device 200 is attached to the body surface of the user 1. An opening 251 is preferably sized to separate the adhesive member 250 from the sensor 231 so that light emitted from the light-emitting unit 231a is not diffusely reflected by the adhesive member 250.

When the sensor 233 is selected from a thermometer, a sphygmomanometer, and an electrocardiograph, the sensor 233 needs to be in contact with the body surface of the user 1. For this reason, it is preferable that the thickness of the sensor 233 and the thickness of the adhesive member 250 from the surface of the biological information measuring device body 210 on which the adhesive member 250 is disposed are substantially equal. In consideration of the elasticity of the adhesive member 250, the thickness H1 of the adhesive member 250 may be set slightly thicker so that the sensor 233 is in close contact with the skin when the biological information measuring device 200 is attached to the body surface of the user 1. In the adhesive member 250, an opening 253 is disposed at a position where the sensor 233 is disposed, but the size of the opening 253 is preferably slightly larger than the size of the sensor 233. Since the size of the opening 253 is slightly larger than the size of the sensor 233, it becomes easy to arrange the adhesive member 250 on the biological information measuring device body 210 at the position of the sensor 233. The configuration of an input unit 220 may be the same as that of the input unit 120, and a detailed description thereof is omitted.

### Modification of Adhesive Member

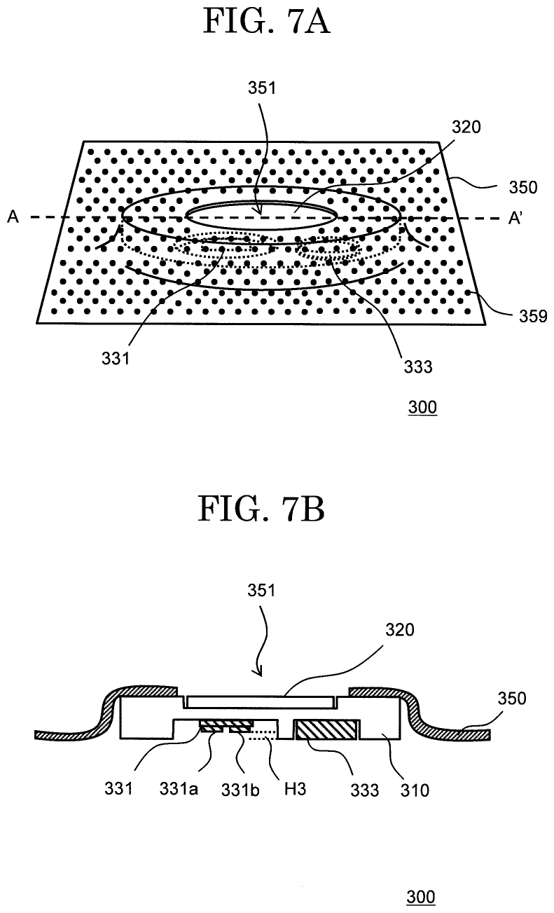

In the above-described embodiment, although the adhesive member is arranged on the same surface as the surface of the biological information measuring device body on which the sensor is arranged is exemplified, the adhesive member may be arranged on the surface of the biological information measuring device body which is opposite to the surface on which the sensor is disposed. FIGS. 7A and 7B are schematic diagrams showing a biological information measuring device 300 according to an embodiment of the present invention. FIG. 7A is a perspective view showing a top surface of the biological information measuring device 300, and FIG. 7B shows a cross-sectional view in a line segment AA′ in FIG. 7A. A biological information measuring device body 310 includes a sensor 331 selected from, for example, a pulsimeter or a reflection-type pulse oximeter and a sensor 333. One or more of measurement devices such as a thermometer, a sphygmomanometer, and an electrocardiograph can be selected and arranged as the sensor 333. The sensor 331 has a light-emitting unit 331a and a light-receiving unit 331b. The configurations of the light-emitting unit 331a and the light-receiving unit 331b may be the same as the configurations of the light-emitting unit 231a and the light-receiving unit 231b, and detailed descriptions thereof are omitted.

It is preferable to arrange the sensor 331 on the biological information measuring device body 310 so that the distance from the light-receiving unit 331b to the skin becomes a distance H3 when the biological information measuring device 300 is attached to the body surface of the user 1. The biological information measuring device body 310 may include only one of the sensor 331 and the sensor 333. In the biological information measuring device body 310, an input unit 320 may be disposed on a surface opposite to the surface on which the sensors 331 and 333 are disposed. The configuration of the input unit 320 may be the same as that of the input unit 120, and a detailed description thereof is omitted.

In the biological information measuring device 300, an adhesive member 350 is disposed on a surface of the biological information measuring device body 310 which is opposite to the surface on which the sensors 331 and 333 are disposed. In the present embodiment, the adhesive member 350 has an adhesiveness on a surface on the same side as the surface on which the sensors 331 and 333 are disposed. The adhesive member 350 may be a known adhesive tape such as a so-called surgical tape. When the input unit 320 is disposed in the biological information measuring device body 310, it is preferable that the adhesive member 350 is not disposed at a position where the input unit 320 is disposed. That is, the adhesive member 350 preferably has an opening 351 at a position corresponding to the input unit 320.

In an embodiment, the adhesive member 350 is preferably a member which does not transmit light. For example, a colorant may be further added to the above material of the adhesive member 350. Light transmittance of the adhesive member 350 is 10% or less, preferably 1% or less, more preferably 0.1% or less.

In an embodiment, the adhesive member 350 may have vents 359. Since the adhesive member 350 has the vents 359, it is possible to suppress the onset of dermatitis due to stuffiness when the adhesive member 350 is attached to the skin. The above adhesive member 250 may be used in combination in the biological information measuring device 300.

### Distribution and Use of Biological Information Measuring Device

The above biological information measuring devices can be repeatedly used by replacing the adhesive member. On the other hand, a biological information measuring devices may be disposable in consideration of hygiene and the like. For example, a physician may prescribe four weeks' worth of biological information measuring devices for one user or one month's worth of biological information measuring devices at a time. In this case, for example, IDs associated with the users may be assigned to a group of biological information measuring devices, and serial numbers corresponding to the number to be distributed may be assigned to each biological information measuring device for management. For example, “1234567” may be assigned to a group of biological information measuring devices to be distributed to the user 1 as an ID, and serial numbers “001” to “100” may be assigned to each of the biological information measuring devices. By managing the group of biological information measuring devices by IDs and serial numbers in this way, the frequency with which the user 1 receives a prescription is reduced to improve convenience, and management in a system using the biological information measuring device becomes easy.

### Modification of Attachment Method

In the above embodiment, an example has been described in which the biological information measuring device is fixed to the body surface of the user 1 only by the adhesive member disposed on the biological information measuring device body, but the method of attaching the biological information measuring device is not limited thereto. For example, by using other member such as a belt in combination, the biological information measuring device body may be fixed to the body surface of the user 1.

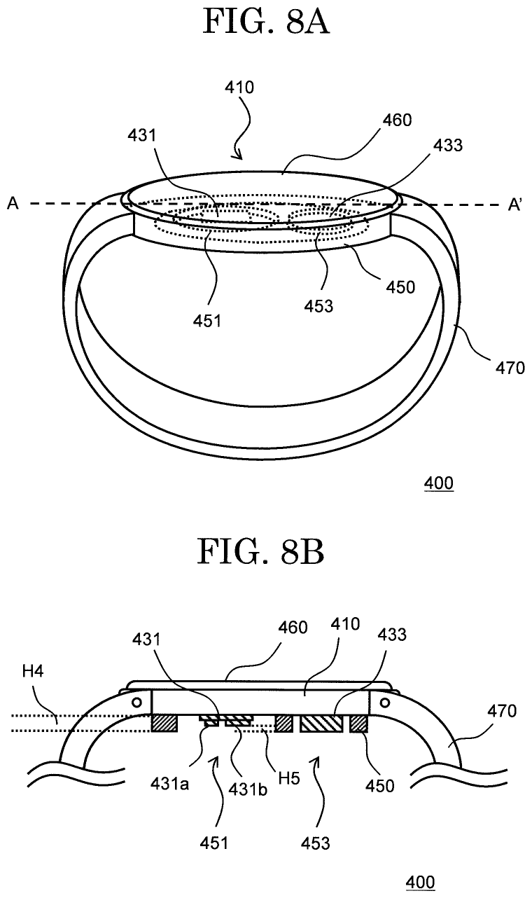

FIGS. 8A and 8B are schematic diagrams showing a biological information measuring device 400 according to an embodiment of the present invention. FIG. 8A is a perspective view showing a top surface of the biological information measuring device 400, and FIG. 8B shows a cross-sectional view in a line segment AA′ of FIG. 8A. For example, a belt 470 which can be fixed to a wrist of the user 1 is connected to a biological information measuring device body 410. That is, the biological information measuring device 400 is a wristwatch-type device. The belt 470 is not particularly limited, and a known belt for a wristwatch can be used, and a detailed description thereof is omitted.

The biological information measuring device body 410 includes a sensor 431 selected from, for example, a pulsimeter or a reflection-type pulse oximeter and a sensor 433. The biological information measuring device 400 includes an adhesive member 450 disposed on the same surface as a surface of the biological information measuring device body 410 on which the sensors 431 and 433 are disposed. One or more of measurement devices such as a thermometer, a sphygmomanometer, and an electrocardiograph can be selected and arranged as the sensor 433. The sensor 431 has a light-emitting unit 431a and a light-receiving unit 431b. The configurations of the light-emitting unit 431a and the light-receiving unit 431b may be the same as the configurations of the light-emitting unit 231a and the light-receiving unit 231b, and detailed descriptions thereof are omitted.

As described above, a thickness H4 of the adhesive member 450 is set so that the light-emitting unit 431a separates from the body surface by a distance H5 required for the light-receiving unit 431b to receive the light irradiated from the light-emitting unit 431a to the skin of the user 1 and reflected through the blood vessel. In consideration of the elasticity of the adhesive member 450, the thickness H5 of the adhesive member 450 may be set slightly thicker so that the distance from the light-receiving unit 431b to the skin becomes the distance H5 when the biological information measuring device 400 is attached to the body surface of the user 1. An opening 451 is preferably sized to separate the adhesive member 450 from the sensor 431 so that light emitted from the light-emitting unit 431a is not diffusely reflected by the adhesive member 450.

In addition, when the sensor 433 is configured to be in contact with the body surface of the user 1, it is preferable that the thickness of the sensor 433 and thickness of the adhesive member 450 from the surface of the biological information measuring device body 410 on which the adhesive member 450 is disposed are substantially equal. In consideration of the elasticity of the adhesive member 450, the thickness H4 of the adhesive member 450 may be set slightly thicker so that the sensor 433 is in close contact with the skin when the biological information measuring device 400 is attached to the body surface of the user 1. In the adhesive member 450, an opening 453 is disposed at a position where the sensor 433 is disposed, but similarly to the opening 253, a size of opening 453 is preferably slightly larger than a size of the sensor 433.

The biological information measuring device 400 may include a display unit 460 on a surface opposite to the surface of the biological information measuring device body 410 on which the sensor 431 and the sensor 433 are disposed. The display unit 460 may be a liquid crystal display or an organic LED display. Instead of the input unit 220, for example, a touch panel may be disposed on the display unit 460.

In the conventional wristwatch-type biological information measuring device, since the biological information measuring device body is fixed to a wrist of the user 1 only by a belt, the biological information measuring device body is deviated from the wrist of the user 1 by operation of the user 1, for example, raising the arm, swinging the arm, or the like, and the measurement position is changed at all times, and it was difficult to measure the accurate biological information. However, in the biological information measuring device 400, it is possible to fix the biological information measuring device body 410 in close contact with the wrist of the user 1 by using the adhesive member 450 and the belt 470 in combination. In the biological information measuring device 400, it is possible to keep the distances between the biological information measuring device body 410 and the body surface of the user 1 constant and acquire high-precision biological information by disposing the adhesive member 450 between the biological information measuring device body 410 and the body surface of the user 1.

In the present embodiment, since the biological information measuring device body 410 is adhered and fixed to the wrist of the user 1 by the adhesive member 450, the distances between the biological information measuring device body 410 and the body surface of the user 1 can be kept constant without tightening the belt 470, and high-precision biological information can be acquired. Therefore, it is not necessary to tighten the belt as in the prior art. Also, a bedridden patient does not need to tighten his/her belt tightly, which helps prevent bedsores.

### Home Medical Support System Using Biological Information Measuring Device

The above biological information measuring device can be applied to various systems utilizing biological information of the user 1. For example, when the user 1 is a patient requiring home nursing, the biological information measuring device can be used in the home medical support system.

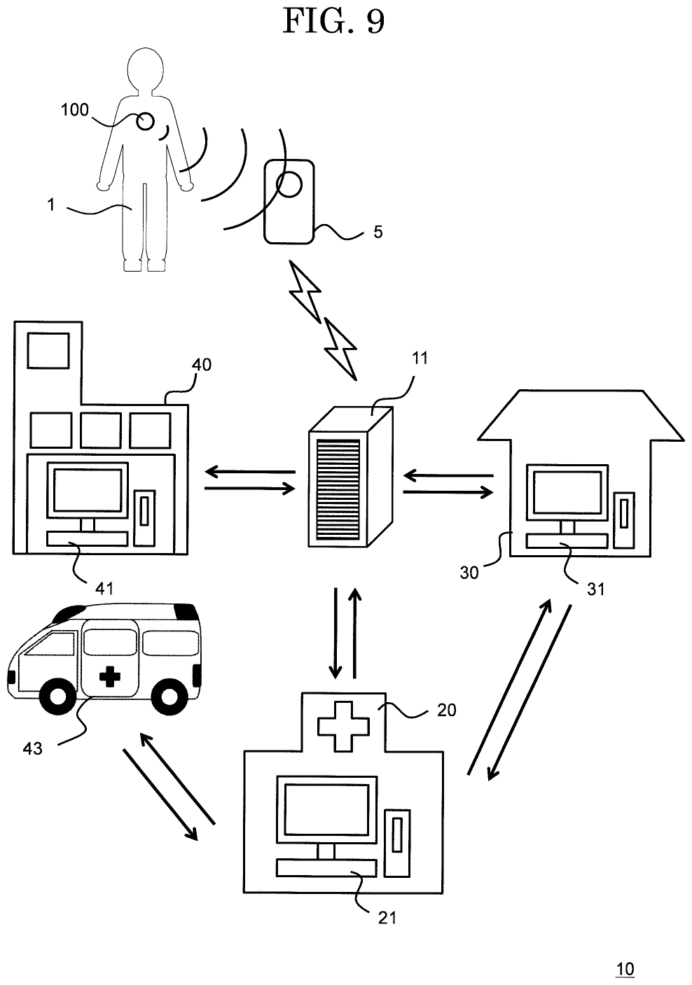

As a specific example of a system using the biological information measuring device 100, a home medical support system 10 will be described. FIG. 9 is a schematic diagram showing the home medical support system 10 according to an embodiment of the present invention. The home medical support system 10 includes the biological information measuring device 100 attached to the user 1, a server 11 capable of communicating with the biological information measuring device 100, and a terminal 21 capable of communicating with the server 11. The server 11 receives the biological information of the user 1 from the biological information measuring device 100. The terminal 21 displays the biological information received via the server 11. In an embodiment, the biological information measuring device 100 and the server 11 may be connected via a line of a dedicated transceiver 5. The biological information measuring device 100 and the dedicated transceiver 5 are connected by, for example, a communication means conforming to a wireless communication standard such as Wi-Fi® or Bluetooth®. The biological information measuring device 100 and the server 11 may be connected via lines of terminals available as a mobile communication system such as a tablet-type terminal such as iPad® or a smartphone owned by a user. In addition, a smartphone other than a smartphone used by the user 1 for a telephone call or the like may be used in place of the dedicated transceiver 5 by dedicating the smartphone to the biological information measuring device 100. In the present embodiment, since the communication with the server 11, which consumes more power, is performed by the dedicated transceiver 5 or a mobile communication system such as a tablet-type terminal or a smart phone owned by the user, the power consumption of the biological information measuring device 100 can be reduced, and the biological information measuring device 100 can be miniaturized and used for a long time.

In an embodiment, the server 11 is, for example, a server connectable to the biological information measuring device 100 via wireless communication and is connected to a medical institution 20, a company 30, and/or the administrative organization 40 via wired communication or wireless communication. That is, the server 11 is a server which functions as a hub for connecting the biological information measuring device 100 and the medical institution 20, the company 30, and/or the administrative organization 40 in the home medical support system 10. A hardware of the server 11 is not particularly limited and is composed of a known server and various electronic devices included in the known server.

In an embodiment, the server 11 may store the information of the user 1 and terminal specification information of the biological information measuring device 100 in association with each other. The information of the user 1 includes the name, address, medical history, and the like of the user 1, and may be information which identifies the user 1. In an embodiment, a storage region of the server 11 for storing the information of the user 1 is preferably a region which is accessible only to an expert, such as medical professionals. In the present embodiment, an expert such as a medical professional can rewrite the information of the user 1 by connecting to the server 11, which is a terminal for medical professionals, via the terminal 21.

When the user 1 uses the biological information measuring device 100, it is preferable to change the position of the body surface to which the biological information measuring device 100 is attached daily to prevent dermatitis. In this case, the measurement sensitivity of the biological information measured may differ depending on the part of the body surface to which the biological information measuring device 100 was attached. For this reason, for example, the server 11 may compare the biological information of the user 1 acquired from the biological information measuring device 100 to the biological information acquired on the previous day and correct it.

The medical institution 20 includes at least a so-called family doctor of the user 1. The medical institution 20 includes various hospitals and clinics. The medical institution 20 includes the terminal 21 for medical professionals and is connected to the server 11 via wired communication or wireless communication. Therefore, the terminal 21 for medical professionals can be connected to the biological information measuring device 100 via the server 11. The terminal 21 for medical professionals may be a general-purpose computer terminal or may be a dedicated terminal. For example, as the terminal 21 for medical professionals, a terminal capable of receiving mail such as a personal computer, a tablet-type terminal, or a smart phone can be used. In an embodiment, the terminal 21 for medical professionals may be connected to the biological information measuring device 100 via the above mobile communication network, rather than via the server 11. The terminal 21 for medical professionals may receive the biological information of the user 1 from the biological information measuring device 100 and allow a medical professional to monitor the biological information of the user 1. When an abnormality is detected in the biological information of the user 1, the biological information measuring device 100 may notify the terminal 21 for medical professionals of the abnormality.

In an embodiment, the terminal 21 for medical professionals may be installed in the plurality of medical institutions 20. Priorities of the terminal 21 for medical professionals to which the abnormality of the biological information of the user 1 is notified from the biological information measuring device 100 (priorities of the medical institution 20 to which the abnormality of the biological information of the user 1 is notified) are registered to the server 11, and when the abnormality is detected in the biological information of the user 1, the server 11 may notify the terminal 21 for medical professionals of the abnormality of the biological information of the user 1 according to the registered priorities. Further, in an embodiment, when there is no response from the first terminal 21 for medical professionals, the abnormality of the biological information of the user 1 may be notified to a terminal capable of receiving a mail such as a tablet-type terminal or a smart phone of the family of the user 1. The terminal 21 for medical professionals may store an application capable of displaying the biological information and the like of the user 1 acquired from the biological information measuring device 100 in a main storage device and execute the application or may display the biological information of the user 1 provided from the server 11 via an internet browser.

In an embodiment, the biological information of the user 1 acquired from the biological information measuring device 100 may be stored in the server 11. The expert such as the medical professionals can browse the biological information of the user 1 stored and accumulated in the server 11 via the terminal 21 for medical professionals. The medical professionals can make a diagnosis and consider a treatment policy at the time of the regular examination of the user 1, based on a medical examination result of the user 1 and the stored biological information of user 1.

The company 30 includes, for example, a nursing facility, a home-visit nursing station, a private security company, and the like. The company 30 includes a terminal 31 for companies and is connected to the server 11 via wired or wireless communication. The terminal 31 for companies may be a general-purpose computer terminal or may be a dedicated terminal. For example, as the terminal 31 for companies, a terminal capable of receiving a mail such as a personal computer, a tablet-type terminal such as iPad®, or a smart phone can be used. In an embodiment, when the terminal 21 for medical professionals is notified of the abnormality of the biological information of the user 1 from the biological information measuring device 100, it is preferable that the medical professionals confirms the biological information of the user 1 and requests the company 30 to support or rescue the user 1.

In an embodiment, the terminal 31 for companies may be connected to the biological information measuring device 100 via the above mobile communication network without the server 11. The terminal 31 for companies may receive the biological information of the user 1 from the biological information measuring device 100 and allow an operator of the company 30 to monitor the biological information of the user 1. When an abnormality is detected in the biological information of the user 1, the biological information measuring device 100 may notify the abnormality to the terminal 31 for companies.

When a request is received from the medical institution 20 or when an abnormality of the biological information of the user 1 is notified to the terminal 31 for companies, the operator of the company 30 can acquire a location information such as the dedicated transceiver 5, or a tablet-type terminal or a smart phone owned by the user 1, and dispatch a support vehicle (for example, a vehicle on which nursing care staff or security guards ride) to a location where the user 1 is located. The terminal 31 for companies may be a general-purpose computer terminal or may be a dedicated terminal. In the home medical support system 10, the terminal 31 for companies may store an application capable of displaying the biological information and the like of the user 1 acquired from the biological information measuring device 100 in the main storage device and execute the application or may display the biological information of the user 1 provided from the server 11 via an internet browser.

Further, in an embodiment, when the user 1 is a user of the company 30, which is a nursing facility, after notifying the abnormality of the user 1 to the terminal 21 for the medical professionals from the biological information measuring device 100, the server 11 may notify the abnormality of the user 1 to the terminal 31 for companies when the terminal 21 for medical professionals did not respond, or when the operation of requesting the support and rescue of the user 1 to the terminal 31 for companies or a terminal 41 for the administrative organization when the terminal 21 for medical professionals did not respond. In this case, the operator of the company 30 can confirm the situation of the user 1 via the terminal 31 for companies and can provide an appropriate support and treatment to the user 1 in cooperation with the medical professionals or under the guidance of the medical professionals. The server 11 may notify the terminal 41 for the administrative organization of the abnormality of the user 1 when the terminal 31 for companies did not respond.

The administrative organization 40 includes, for example, but is not limited to, a fire station (including a fire department) and a police station (including a police department). The administrative organization 40 includes the terminal 41 for the administrative organization and is connected to the server 11 via wired communication or wireless communication. The terminal 41 for the administrative organization may be a general-purpose computer terminal or may be a dedicated terminal. For example, as the terminal 41 for the administrative organization, a terminal capable of receiving mail such as a personal computer, a tablet-type terminal such as iPad®, or a smart phone can be used. In an embodiment, when the terminal 21 for medical professionals is notified of the abnormality of the biological information of the user 1 from the biological information measuring device 100, it is preferable that the medical professionals confirms the biological information of the user 1 and requests the administrative organization 40 to support or rescue the user 1.

Further, in an embodiment, the terminal 41 for the administrative organization may be connected to the biological information measuring device 100 via the above mobile communication network without the server 11. The terminal 41 for the administrative organization may receive the biological information of the user 1 from the biological information measuring device 100 and allow an operator of the administrative organization 40 to monitor the biological information of the user 1. When an abnormality is detected in the biological information of the user 1, the biological information measuring device 100 may notify the abnormality to the terminal 41 for the administrative organization.

When the operator of the administrative organization 40 receives a request from the medical institution 20, or when the abnormality of the biological information of the user 1 is notified to the terminal 41 for the administrative organization, the operator of the administrative organization 40 can acquire a location information such as the dedicated transceiver 5, or a tablet-type terminal or a smart phone owned by the user 1, and dispatch an ambulance 43 or patrol car (not shown) to a location where the user 1 is located. The terminal 41 for the administrative organization may be a general-purpose computer terminal or may be a dedicated terminal. In the home medical support system 10, the terminal 41 for the administrative organization may store an application capable of displaying the biological information of the user 1 acquired from the biological information measuring device 100 in the main storage device and execute the application or may display the biological information of the user 1 provided from the server 11 via an internet browser.

### Safety Assistance System Using Biological Information Measuring Device

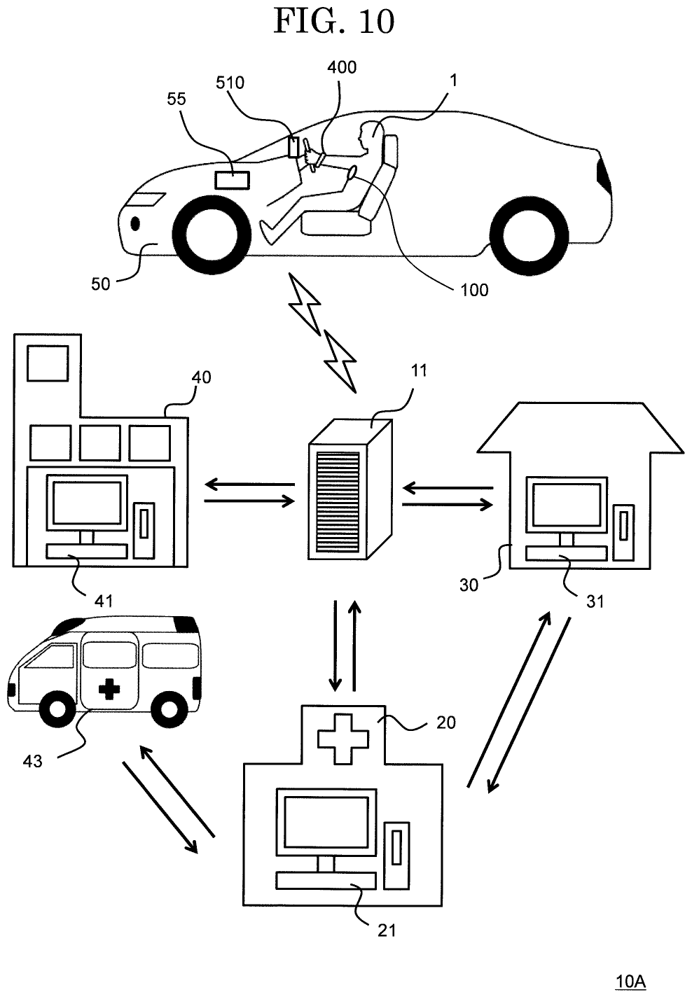

In the above embodiment, the home medical support system has been described, but an example in which the above biological information measuring devices are used in a safe driving assistance system when the user 1 drives the vehicle will be described. FIG. 10 is a schematic diagram showing a safe driving assistance system 10A according to an embodiment of the present invention. The safe driving assistance system 10A includes, for example, the vehicle 50 on which the user 1 drives or rides, the server 11, the medical institution 20, the company 30, and the administrative organization 40, but is not limited thereto. The server 11, the medical institution 20, the company 30, and the administrative organization 40 may have the same configurations as those of the above-described embodiments, and detailed descriptions thereof are omitted.

The vehicle 50 includes an in-vehicle wireless communication means, and biological information measuring device 100 and the server 11 can communicate with each other via wireless communication. As the wireless communication, for example, a mobile communication network can be used. In an embodiment, the biological information measuring device 100 may be connected to the server 11 via a mobile phone, a smart phone, or a tablet-type terminal such as iPad® owned by the user 1, or a dedicated transceiver. Alternatively, the vehicle 50 may be equipped with a dedicated wireless communication device connectable to a mobile communication network.

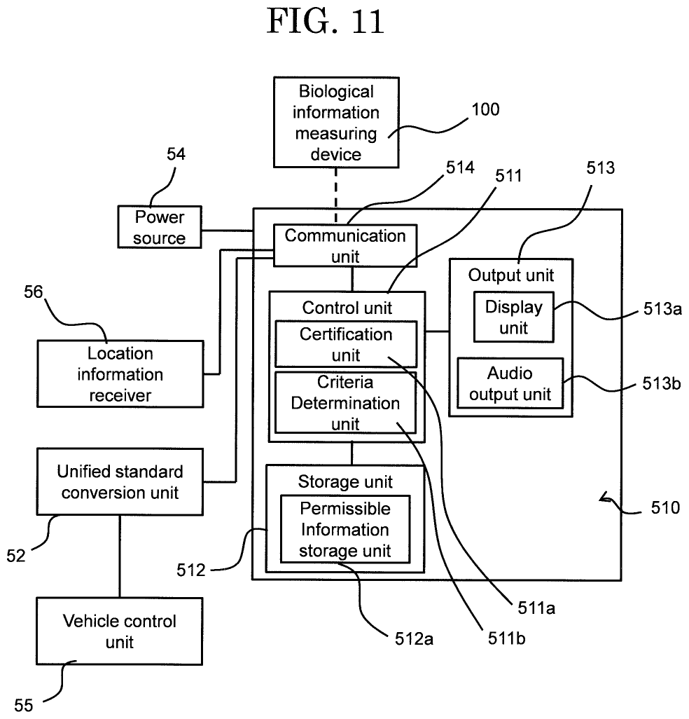

In an embodiment, the vehicle 50 is equipped with a safe driving assistance in-vehicle device 510 and the safe driving assistance in-vehicle device 510 is connected to a vehicle control unit 55 of the vehicle 50. FIG. 11 is a block diagram showing the safe driving assistance in-vehicle device 510 according to an embodiment of the present invention. The safe driving assistance in-vehicle device 510 includes, for example, a control unit 511 including a certification unit 511a and a criteria determination unit 511b, a storage unit 512 including a permissible information storage unit 512a, an output unit 513 including a display unit 513a and an audio output unit 513b, and a communication unit 514. The safe driving assistance in-vehicle device 510 is supplied with power from a power source 54 of the vehicle 50. The power source 54 is, for example, a battery mounted on the vehicle 50. In an embodiment, the safe driving assistance in-vehicle device 510 is connected to the biological information measuring device 100 via a communication unit 34. Also, in an embodiment, the safe driving assistance in-vehicle device 510 is connected to a location information receiver 56. In an embodiment, the safe driving assistance in-vehicle device 510 is preferably connected to the vehicle control unit 55 via a unified standard conversion unit 52.

In an embodiment, the control unit 511 is a device for controlling the safe driving assistance in-vehicle device 510, and is, for example, a central processing unit (CPU). The control unit 511 includes a program for controlling the safe driving assistance in-vehicle device 510. The program for controlling the safe driving assistance in-vehicle device 510 is stored in the storage unit 512 and executed by the control unit 511. In an embodiment, the control unit 511 may include an operating system (OS) which controls the safe driving assistance in-vehicle device 510 and an application program or module which functions in the safe driving assistance system 10A.

In an embodiment, the control unit 511 includes the certification unit 511a and the criteria determination unit 511b. The certification unit 511a is an application program or module for certificating the user 1. The certification unit 511a can specify the user 1 based on the physical address assigned to the communication unit 117 of the biological information measuring device 100 or the physical address assigned to a communication unit of the dedicated transceiver 5. When the certification information of the user 1 includes a code or the like indicating that the user 1 is permitted to drive a vehicle, the certification unit 511a may permit the vehicle control unit 55 to drive the vehicle 50 by the user 1. If the certification information of the user 1 does not include a code or the like indicating that the user 1 is allowed to drive a vehicle, even if the user 1 operates to start the engine of the vehicle 50, the engine starting operation may be shut off via the vehicle control unit 55.

In an embodiment, the criteria determination unit 511b is an application program or module for comparing the permissible information of the user 1 stored in the server 11 or the biological information measuring device 100 with at least the biological information measured by the biological information measuring device 100 to control the operating condition of the vehicle 50. The permissible information of the user 1 is set by a medical professional and stored in the server 11 or the biological information measuring device 100. In an embodiment, the safe driving assistance in-vehicle device 510 may receive the permissible information of the user 1 from the server 11 or the biological information measuring device 100 via the communication unit 34 and store the received permissible information in the permissible information storage unit 512a.

In an embodiment, the criteria determination unit 511b compares the permissible information of the user 1 to the measured biological information. For example, when it is stored that the heart rate is less than a predetermined number as the permissible information of the user 1 stored in the permissible information storage unit 512a, the criteria determination unit 511b transmits a signal for not transmitting a special signal to the vehicle control unit 55 or for permitting the vehicle to be driven if the heart rate of the user 1 measured by the biological information measuring device 100 is less than the predetermined number. On the other hand, when the heart rate of the user 1 measured by the biological information measuring device 100 is equal to or higher than the predetermined number, the criteria determination unit 511b may transmit a signal for displaying an alert and outputting sounds to the output unit 513. When the heart rate of the user 1 is equal to or higher than a preset risk value, the criteria determination unit 511b transmits a signal for stopping the vehicle 50 to the vehicle control unit 55.

In an embodiment, the output unit 513 includes, for example, the display unit 513a and the audio output unit 513b. The display unit 513a is, for example, a display device such as a display or a heads-up display. Further, the audio output unit 513b may be an in-vehicle speaker or a separate speaker disposed in the safe driving assistance in-vehicle device 510. The display unit 513a and the audio output unit 513b can output video and audio for calling attention to the user 1 and output videos and audios for warning the user 1 in accordance with signals received from the criteria determination unit 511b. In addition, the audio output unit 513b may output audios to alert the surroundings to the outside of the vehicle when the vehicle 50 is stopped urgently.

In an embodiment, the communication unit 514 includes, but is not limited to, a communication means conforming to a wireless communication standard such as Wi-Fi® or Bluetooth® for wireless communication with the biological information measuring device 100. The communication unit 514 may be connected to the biological information measuring device 100 via the dedicated transceiver 5.

The vehicle control unit 55 is a control device which controls the vehicle 50 and is a computer or module mounted on the vehicle 50. Since the vehicle control unit 55 has a known vehicle control mechanism, a detailed description thereof will be omitted. In an embodiment, the vehicle control unit 55 is connected to the safe driving assistance in-vehicle device 510 and controls the vehicle 50 based on a signal received from the safe driving assistance in-vehicle device 510 in addition to the driving operation performed by the user 1. In an embodiment, the vehicle control unit 55 includes safe driving support techniques such as automatic braking, pedaling misapplication prevention, lane departure warning, and cruise control.

Since the vehicle control unit 55 is a control device specific to the vehicle 50, it has a different configuration for each automaker or vehicle type. When the safe driving assistance in-vehicle device 510 is connected directly to the vehicle control unit 55, the safe driving assistance in-vehicle device 510 must be configured for the vehicle control unit 55, which is different for each automaker or vehicle type. Alternatively, the vehicle control unit 55 may need to be altered to allow the vehicle control unit 55 to use the signal received from the safe driving assistance onboarding-vehicle device 510.

The unified standard conversion unit 52 is a computer or module that converts the signal output by the safe driving assistance in-vehicle device 510 for the vehicle control unit 55 which is different for each automaker or vehicle type. In an embodiment, it is preferable to dispose the unified standard conversion unit 52 to connect the safe driving assistance in-vehicle device 510 and the vehicle control unit 55. Therefore, the unified standard conversion unit 52 is preferably a device configured by a unified standard of the automobile industry capable of controlling the vehicle control unit 55 which is different for each automaker or vehicle type.

The location information receiver 56 may be a known car navigation system. In an embodiment, the location information receiver 56 acquires location information of the vehicle 50, traffic volume information, and map information in the vicinity of the point where the vehicle 50 travels and transmits the information to the safe driving assistance in-vehicle device 510, but the present invention is not limited thereto.

In this manner, it is possible to support the safe driving of the vehicle 50 by the user 1 based on the biological information acquired from the biological information measuring device 100. In place of the biological information measuring device 100, any of the above-described biological information measuring devices can be used. In the safe driving assistance system 10A, for example, the wristwatch-type biological information measuring system 400 may be used.

In the safe driving assistance system 10A, the safe driving assistance in-vehicle device 510 can be connected to the medical institution 20, the company 30, and the administrative organization 40 via the server 11. When there is an abnormality in the physical condition of the user 1 during driving of the vehicle 50, rescue and support of the user 1 can be performed in cooperation with the medical institution 20, the company 30, and the administrative organization 40 as described in the home medical support system 10. The method of rescuing and supporting the user 1 in the safe driving assistance system 10A may be the same as the method of rescuing and supporting the user 1 in the home medical support system 10, and detailed descriptions thereof are omitted.

By using the biological information measuring device capable of measuring the biological information of the user 1 with high accuracy, it is possible to detect the abnormality of the user 1 with high accuracy, and it is possible to perform driving assistance with high safety in the safe driving assistance system 10A.

### Health Management System Using Biological Information Measuring Device

Although the above home medical support system 10 and the safe driving assistance system 10A are systems focusing on rescuing and supporting the user 1 when the abnormality occurs in the biological information of the user 1, the biological information measuring device according to the embodiment of the present invention can be applied to health management of a healthy person.

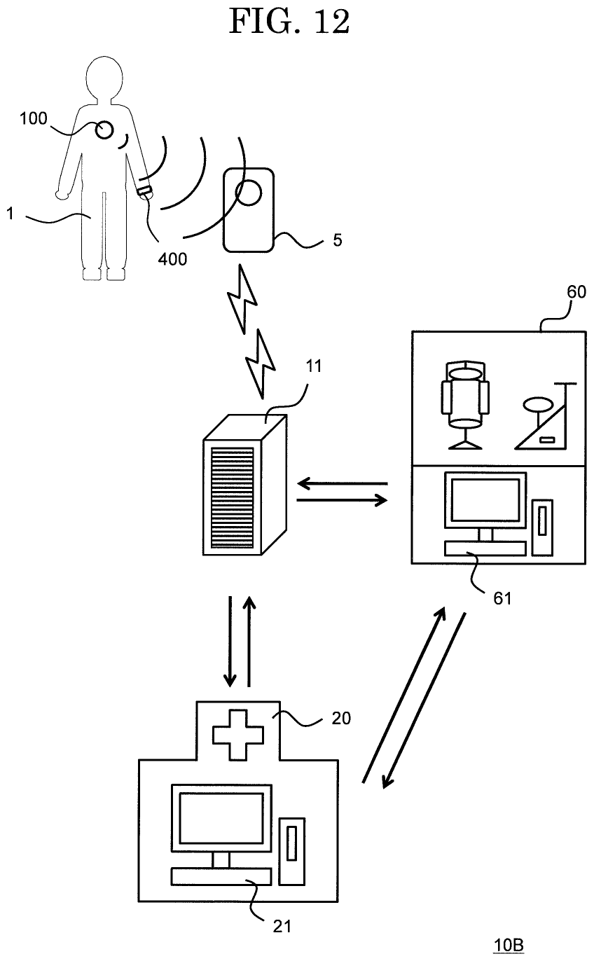

FIG. 12 is a schematic diagram showing a health management system 10B according to an embodiment of the present invention. The health management system 10B includes, for example, the biological information measuring device 100 worn by the user 1, the server 11, a medical institution 20, and a gym 60, but is not limited thereto. The server 11 and the medical institution 20 may have the same configuration as that of the above embodiment, and a detailed description thereof will be omitted. The biological information measuring device 100 may be connected to the server 11 via the dedicated transceiver 5. In addition, any of the above biological information measuring devices can be used instead of the biological information measuring device 100. In the health management system 10B, for example, the wristwatch-type biological information measuring device 400 may be used.

The sports gym 60 is a facility normally used by the user 1 and is a known facility, and includes, for example, a terminal 61 for gyms and is connected to the server 11 via wired communication or wireless communication. The terminal 61 for gyms may be a general-purpose computer terminal or may be a dedicated terminal. For example, as the terminal 61 for gyms, a terminal capable of receiving mail such as a personal computer, a tablet-type terminal such as iPad®, or a smart phone can be used. In an embodiment, the terminal 61 for gyms may be connected to the biological information measuring device 100 via the above mobile communication network without the server 11. The terminal 61 for gyms may receive the biological information of the user 1 from the biological information measuring device 100 and allow an operator of the terminal 61 for gyms to monitor the biological information of the user 1.

In the health management system 10B, the terminal 61 for gyms may store an application capable of displaying the biological information of the user 1 acquired from the biological information measuring device 100 in the main storage device and execute the application or may display the biological information of the user 1 provided from the server 11 via an internet browser.

In the health management system 10B, by detecting the biological information of the user 1 with high accuracy, the medical institution 20 and the sport gym 60 can cooperate with each other to perform health management of the user 1. For example, the medical institution 20 can give instructions and advice to the gym 60 on the training method of the user 1 based on the biological information acquired from the user 1. Training is done to promote health, but excessive training can be a burden on the user 1 and can also lead to injuries and illnesses. In particular, excessive training may be dangerous for the user 1 with chronic disease. Therefore, it is beneficial to promote the health of the user 1 when the medical institution 20 and the sport gym 60 cooperate with each other to perform health management of the user 1.

In the health management system 10B, by continuously acquiring the biological information of the user 1, the biological information of the user 1 can be used for improving the lifestyle of the user 1 or preventing a disease. In addition, by using the biological information measuring device capable of measuring biological information of the user 1 with high accuracy, it is possible to accurately grasp the health condition of the user 1, and it is possible to realize the health management of the user 1 for preventing lifestyle-related diseases.

According to an embodiment of the present invention, there is provided a biological information measuring device having wearability capable of obtaining high measurement accuracy. Further, according to an embodiment of the present invention, there is provided a biological information measuring device having wearability for improving QOL of a user.

What is claimed is:

1. A biological information measuring device comprising:
a body;
a first sensor and a second sensor disposed in a first surface of the body; and
an adhesive member having one or more openings;
wherein each of the first sensor and the second sensor are disposed so as to be surrounded by one of the one or more openings and separated from the adhesive member in the surrounding opening;
wherein measuring surfaces of the first sensor and the second sensor are exposed;
wherein the adhesive member is attached to skin when the biological information measuring device is mounted on the skin;
wherein an end part of the first sensor is separated from the skin; and
the second sensor is in contact with the skin.

2. The biological information measuring device according to claim 1, wherein:
the adhesive member is disposed on the first surface of the body;
the thickness of the adhesive member from the first surface of the biological information measuring device is greater than the thickness from the first surface to the end part of the first sensor; and
the thickness of the adhesive member from the first surface and the thickness from the first surface to the end part of the second sensor is substantially the same.

3. The biological information measuring device according to claim 1, wherein:
the adhesive member is disposed on a second surface opposite to the first surface to overlap the body; and
the adhesive member has an adhesiveness on the same side as the first surface.

4. The biological information measuring device according to claim 3, wherein the adhesive member has vents.

5. The biological information measuring device according to claim 1, wherein the adhesive member contains one or more additive agents selected from a group consisting of an antibacterial agent, an aromatic agent, capsaicin, and methyl salicylate.

6. The biological information measuring device according to claim 1, further comprising an input unit and a communication unit, wherein the input unit is disposed on a second surface opposite to the first surface.

7. The biological information measuring device according to claim 1, wherein:
the biological information measuring device is attached to an abdomen of a user; and
the biological information measuring device is supplied with power from a belt worn by the user.

8. The biological information measuring device according to claim 1, wherein:
the biological information measuring device is attached to the back of a user; and
the biological information measuring device is supplied with power from a seat occupied by the user.
